(19) United States
(12) Patent Application Publication
(10) Pub. No.: US 2003/0119010 A1
(43) Pub. Date: Jun. 26, 2003

(54) Polynucleotides and polypeptides linked to a disease or condition

(75) Inventors: Elizabeth Ellen Powell, Queensland (AU); Julie Ruth Jonsson, Queensland (AU)

Correspondence Address: AKIN, GUMP, STRAUSS, HAUER & FELD, L.L.P., ONE COMMERCE SQUARE, SUITE 2200, 2005 MARKET STREET, PHILADELPHIA, PA, 19103, US

(73) Assignee: University of Queensland, Queensland (AU)

(21) Appl. No.: 10/087,268

(22) Filed: Mar. 1, 2002

Related U.S. Application Data
(60) Provisional application No. 60/272,398, filed on Mar. 1, 2001.

(51) Int. Cl. C12Q 1/68; G01N 33/53

(52) U.S. Cl. 435/6; 435/7.1

(57) ABSTRACT

The invention relates to the discovery that alleles of the transforming growth factor (beta) (TGF-(beta)) gene that are associated with high levels of expression or high functional activity of the protein than are other alleles can be correlated with development, progression, or both, of fibrotic conditions other than lung fibrosis. The invention also relates to other alleles associated with development, progression, or both, of fibrotic conditions.

### CROSS-REFERENCE TO RELATED APPLICATIONS

This application is entitled to priority, pursuant to 35 U.S.C 119(e) to U.S. Provisional Patent Application No. 60/272,398, filed Mar. 1, 2001.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

This invention relates generally to polynucleotides and polypeptides linked to fibrotic conditions.

Bibliographic details of the publications numerically referred to in this specification are collected at the end of the description.

Progressive hepatic fibrosis and cirrhosis develops in 20-30% of patients with chronic hepatitis C(HCV). For those patients who fail to respond to anti-viral therapy, there are currently no approved therapeutic options designed to delay or reverse the progression of fibrosis.

In epidemiological studies of chronic HCV infection, age, duration of infection, alcohol consumption, and male gender are independent factors related to histological severity (1). We have also recently demonstrated a significant association between increased body mass index and steatosis and fibrosis in chronic HCV (2). However, in any individual, the factors that determine increasing fibrosis and progressive disease remain unknown. Cytokines secreted in response to cell injury have a central role in the pathogenesis of liver fibrosis (3). The most dominant fibrogenic cytokine in hepatic fibrosis is transforming growth factor-(beta)1 (TGF-(beta) 1), which contributes to the activation of stellate cells and their production of extracellular matrix proteins (4). TGF-(beta)1 mRNA is increased in the liver of patients with chronic HCV relative to healthy controls and the level of expression has been shown to correlate with expression of type 1 collagen mRNA (5). Stellate cells also produce anti-inflammatory cytokines, in particular interleukin-10 (IL-10), which has prominent anti-fibrotic activity by down-regulating collagen 1 expression while up-regulating interstitial collagenase (6). Other cytokines may be both pro-and/or anti-fibrotic, depending on the predominant cell type responding to their effects. In addition to a major role as a mediator of the inflammatory response, tumor necrosis factor-alpha (TNF-(alpha)) down-regulates collagen synthesis and may promote apoptosis of either inflammatory or fibrogenic cells (7).

The capacity for cytokine production in individuals has a major genetic component. There are striking differences between individuals in their ability to produce cytokines following in vitro stimulation of peripheral blood leukocytes. This has been ascribed to polymorphisms within the regulatory regions or signal sequences of cytokine genes. Several polymorphic sites have been described within the TGF-(beta)1 gene, including two in the promoter region at positions −800 and −509 base pairs (bp) from the transcription start site, one at position +72 bp in a non-translated region, and two in the signal sequence at codons 10 and 25 (8). The promoter region of the IL-10 gene contains three biallelic polymorphisms at positions −1082, −819, and −592 bp from the transcription start site, that produce three different haplotypes, GCC, ACC and ATA (9). Similarly, the promoter region of the TNF-(alpha) gene contains a biallelic polymorphism at position −308 bp from the transcription start site (10). Inheritance of the high TGF-(beta)1-producing genotype is associated with the development of fibrotic lung disease (8). However the role of these genetic polymorphisms in the progression of other fibrotic diseases has not yet been evaluated.

In cardiac and renal fibrosis, TGF-(beta)1 production may be enhanced by angiotensin II (AII), the principal effector molecule of the renin-angiotensin system (RAS) (11,12). The primary action of the RAS is to regulate vascular tone and renal salt excretion. However recent data indicate that, independently of its effects on blood pressure, AII may augment the accumulation of extracellular matrix (13). Functional polymorphisms of genes of the RAS have been described, including a nucleotide substitution at 6 bp from the transcription start site in the promoter of angiotensinogen (AT), the precursor peptide for angiotensin I, and an absence deletion (D/I) within intron 16 of the angiotensin 1-converting enzyme (ACE), which converts AI to AII (14,15). These polymorphisms have been postulated to contribute to inter-individual variability in the outcome of various renal and cardiovascular diseases. However their contribution to progressive fibrosis in other organs has not been evaluated.

### BRIEF SUMMARY OF THE INVENTION

The present invention is predicated in part on the discovery that TGF-(beta) alleles, which permit TGF-(beta) to be produced at a higher level and/or functional activity than other TGF-(beta) alleles, correlate with the development and/or progression of fibrotic conditions other than lung fibrosis. The inventors have also discovered that alleles of a gene member of the RAS, which permit AII to be produced at a higher level and/or functional activity than other alleles of said gene member, also correlate with the development and/or progression of fibrotic conditions. The foregoing discoveries have been reduced to practice inter alia in methods for diagnosing the presence of, or a predisposition to develop, a fibrotic condition, and in compositions for treating and/or preventing a fibrotic condition, as described hereinafter.

Accordingly, in one aspect of the present invention, there is provided a method for diagnosing the presence of, or a predisposition to develop, a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, and wherein said allele permits the production of a TGF-(beta) polypeptide at a level and/or functional activity that correlates with the development of said condition.

Preferably, said allele is an allele of the TGF-(beta)1 gene.

Suitably, said allele is an allele of a gene member of the renin-angiotensin system (RAS). Preferably, said allele of said gene member permits angiotensin II (AII) to be produced at a level sufficient to induce the production of TGF-(beta)1 at said level and/or functional activity. More preferably, said allele of said gene member is an angiotensinogen (AT) allele.

In another aspect, the invention resides in a method for diagnosing a predisposition to develop a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, and wherein said allele permits the production of a TGF-(beta) polypeptide at a level and/or functional activity that correlates with the development of said condition.

In yet another aspect, the invention contemplates a method for diagnosing a predisposition to develop hepatic fibrosis in a patient, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, and wherein said allele permits the production of a TGF-(beta) polypeptide at a level and/or functional activity that correlates with the development of said condition.

In still another aspect, the invention extends to a method for diagnosing a predisposition to develop progressive hepatic fibrosis in a patient, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, and wherein said allele permits the production of a TGF-(beta) polypeptide at a level and/or functional activity that correlates with the development of said condition.

According to a further aspect, the invention provides a method for diagnosing a higher risk of developing a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, wherein said allele correlates with said higher risk.

Suitably, said allele is an allele of the TGF-(beta)1 gene. Preferably, said TGF-(beta)1 allele comprises the sequence set forth in SEQ ID NO: 1.

Suitably, said allele of said gene, which belongs to the same regulatory or biosynthetic pathway as the TGF-(beta) gene, is an allele of a gene member of the RAS. In a preferred embodiment of this type, the allele of said gene member is an AT allele.

Preferably, said AT allele that correlates with said higher risk comprises the sequence set forth in any one of SEQ ID NO: 3.

In yet a further aspect, the invention contemplates a method for diagnosing a lower risk of developing a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, wherein said allele correlates with said lower risk.

Suitably, the allele of said TGF-(beta) gene that correlates with said lower risk is a TGF-(beta)1 allele, which preferably comprises the sequence set forth in SEQ ID NO: 4.

Suitably, said allele of said gene, which belongs to the same regulatory or biosynthetic pathway as the TGF-(beta) gene, is an allele of a gene member of the RAS. In a preferred embodiment of this type, the allele of said gene member is an AT allele. Preferably, said AT allele that correlates with said lower risk comprises the sequence set forth in any one of SEQ ID NO: 6.

According to another aspect, the invention provides a method for diagnosing a higher risk of developing a fibrotic condition in a patient, wherein said fibrotic condition is other that lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein each of said different alleles correlates with said higher risk.

In another aspect, the invention resides in a method for diagnosing a lower risk of developing a fibrotic condition in a patient, wherein said fibrotic condition is other that lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein each of said different alleles correlates with said lower risk.

In yet another aspect, the invention extends to a method for diagnosing an intermediate risk of developing a fibrotic condition in a patient, wherein said fibrotic condition is other than lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein at least one of said different alleles correlates with a higher risk of developing said condition and wherein at least one other of said different alleles correlates with a lower risk of developing said condition.

In yet another aspect, the invention provides a method for diagnosing the presence of, or a predisposition to develop, a fibrotic condition in a patient, comprising detecting in a biological sample obtained from said patient a target molecule selected from an allele of a gene belonging to the same regulatory or biosynthetic pathway as a TGF-(beta) gene and an expression product of said allele, wherein said allele permits the production of a TGF-(beta) polypeptide at a level and/or functional activity that correlates with the development of said condition.

In a further aspect, the invention extends to a method for diagnosing the presence of, or a predisposition to develop, a fibrotic condition in a patient, comprising detecting in a biological sample obtained from said patient a level and/or functional activity of an expression product of a gene belonging to the same regulatory or biosynthetic pathway as a TGF-(beta) gene, wherein said level and/or functional activity correlates with the development of said condition.

In another aspect, the invention resides in a method for diagnosing a predisposition to develop a fibrotic condition in a patient, comprising detecting in a biological sample obtained from said patient a target molecule selected from an allele of a gene belonging to the same regulatory or biosynthetic pathway as a TGF-(beta) gene and an expression product of said allele, wherein said allele permits the production of a TGF-(beta) polypeptide at a level and/or functional activity that correlates with the development of said condition.

In yet another aspect, the invention provides a method for diagnosing a predisposition to develop a fibrotic condition in a patient, comprising detecting in a biological sample obtained from said patient a level and/or functional activity of an expression product of a gene belonging to the same regulatory or biosynthetic pathway as a TGF-(beta) gene, wherein said level and/or functional activity correlates with the development of said condition.

According to a further aspect, the invention provides a method for diagnosing a higher risk of developing a fibrotic condition in a patient, comprising detecting in a biological sample obtained from said patient a target molecule selected from an allele of a gene belonging to the same regulatory or biosynthetic pathway as a TGF-(beta) gene and an expression product of said allele, wherein said allele correlates with said higher risk.

Suitably, said allele that correlates with said higher risk is an AT allele, which preferably comprises the sequence set forth in any one of SEQ ID NO: 3.

In yet a further aspect, the invention contemplates a method for diagnosing a lower risk of developing a fibrotic condition in a patient, comprising detecting in a biological sample obtained from said patient a target molecule selected from an allele of a gene belonging to the same regulatory or biosynthetic pathway as a TGF-(beta) gene and an expression product of said allele, wherein said allele correlates with said lower risk.

Suitably, said allele that correlates with said lower risk is an AT allele, which preferably comprises the sequence set forth in any one of SEQ ID NO: 6.

According to another aspect, the invention provides a method for diagnosing a higher risk of developing a fibrotic condition in a patient, comprising detecting in a biological sample obtained from said patient at least two target molecules selected from different alleles of a gene belonging to the same regulatory or biosynthetic pathway as a TGF-(beta) gene or expression products thereof, wherein each of said alleles correlates with a higher risk of developing said condition.

In another aspect, the invention resides in a method for diagnosing a lower risk of developing a fibrotic condition in a patient, comprising detecting in a biological sample obtained from said patient at least two target molecules selected from different alleles of a gene belonging to the same regulatory or biosynthetic pathway as a TGF-(beta) gene or expression products thereof, wherein each of said alleles correlates with a lower risk of developing said condition.

In yet another aspect, the invention extends to a method for diagnosing an intermediate risk of developing a fibrotic condition in a patient, comprising detecting in a biological sample obtained from said patient at least two target molecules selected from different alleles of a gene belonging to the same regulatory or biosynthetic pathway as a TGF-(beta) gene or expression products thereof, wherein at least one of said different alleles correlates with a higher risk of developing said condition and wherein at least one other of said different alleles correlates with a lower risk of developing said condition.

In another aspect, the invention encompasses a method for diagnosing a lower risk of developing a fibrotic condition in a patient, comprising detecting in a biological sample obtained from said patient a level and/or functional activity of an expression product of a gene belonging to the same regulatory or biosynthetic pathway as a TGF-(beta) gene, wherein said level and/or functional activity correlates with a lower risk of developing said condition.

In yet another aspect, the invention provides a method for diagnosing the presence of, or a predisposition to develop, a fibrotic condition in a patient, comprising detecting in a biological sample obtained from said patient a target molecule selected from an allele of a gene belonging to the renin-angiotensin system (RAS), and an expression product of said allele, wherein said allele is associated with the development of said condition.

In a further aspect, the invention extends to a method for diagnosing the presence of, or a predisposition to develop, a fibrotic condition in a patient, comprising detecting in a biological sample obtained from said patient a level and/or functional activity of an expression product of a gene belonging to the renin-angiotensin system (RAS), wherein said level and/or functional activity correlates with the development of said condition.

In another aspect, the invention resides in a method for diagnosing a predisposition to develop a fibrotic condition, comprising detecting in a biological sample obtained from said patient a target molecule selected from an allele of a gene belonging to the renin-angiotensin system (RAS) and an expression product of said allele, wherein said allele correlates with the development of said condition.

In yet another aspect, the invention provides a method for diagnosing a predisposition to develop a fibrotic condition in a patient, comprising detecting in a biological sample obtained from said patient a level and/or functional activity of an expression product of a gene belonging to the renin-angiotensin system (RAS), wherein said level and/or functional activity correlates with the development of said condition.

According to a further aspect, the invention provides a method for diagnosing a higher risk of developing a fibrotic condition in a patient, comprising detecting in a biological sample obtained from said patient a target molecule selected from an allele of a gene belonging to the renin-angiotensin system (RAS) and an expression product of said allele, wherein said allele correlates with said higher risk.

Suitably, said allele that correlates with said higher risk is an AT allele, which preferably comprises the sequence set forth in any one of SEQ ID NO: 3.

In yet a further aspect, the invention contemplates a method for diagnosing a lower risk of developing a fibrotic condition in a patient, comprising detecting in a biological sample obtained from said patient a target molecule selected from an allele of a gene belonging to the renin-angiotensin system (RAS) and an expression product of said allele, wherein said allele correlates with said lower risk.

Suitably, said allele that correlates with said lower risk is an AT allele, which preferably comprises the sequence set forth in any one of SEQ ID NO: 6.

According to another aspect, the invention provides a method for diagnosing a higher risk of developing a fibrotic condition in a patient, comprising detecting in a biological sample obtained from said patient at least two target molecules selected from different alleles of a gene belonging to the renin-angiotensin system (RAS) or expression products thereof, wherein each of said different alleles correlates with a higher risk of developing said condition.

In another aspect, the invention resides in a method for diagnosing a lower risk of developing a fibrotic condition in a patient, comprising detecting in a biological sample obtained from said patient at least two target molecules selected from different alleles of a gene belonging to the renin-angiotensin system (RAS) or expression products thereof, wherein each of said different alleles correlates with a lower risk of developing said condition.

In yet another aspect, the invention extends to a method for diagnosing an intermediate risk of developing a fibrotic condition in a patient, comprising detecting in a biological sample obtained from said patient at least two target molecules selected from different alleles of a gene belonging to the renin-angiotensin system (RAS) or expression products thereof, wherein at least one of said different alleles correlates with a higher risk of developing said condition and wherein at least one other of said different alleles correlates with a lower risk of developing said condition.

In another aspect, the invention encompasses a method for diagnosing a lower risk of developing a fibrotic condition in a patient, comprising detecting in a biological sample obtained from said patient a level and/or functional activity of an expression product of a gene belonging to the renin-angiotensin system (RAS), wherein said level and/or functional activity correlates with a lower risk of developing said condition.

The invention also extends to the use of a TGF-(beta) polynucleotide corresponding to a TGF-(beta) allele, or transcript thereof, that correlates with the development of a fibrotic condition, or a polynucleotide corresponding to an allele of a gene belonging to the same regulatory or biosynthetic pathway as a TGF-(beta) gene, or transcript thereof, which allele correlates with the development of a fibrotic condition, or a polypeptide encoded by said TGF-(beta) polynucleotide, or an antigen-binding molecule that is immuno-interactive specifically with TGF-(beta) polynucleotide, for diagnosing the presence of, or a predisposition to develop, a fibrotic condition.

In yet another aspect, the invention extends to the use of an agent, which modulates the level and/or functional activity of an expression product of an allele selected from an allele of a TGF-(beta) gene and an allele of a gene belonging to the same regulatory or biosynthetic pathway as a TGF-(beta) gene, in the manufacture of a medicament for treating and/or preventing a fibrotic condition, wherein said agent has been identified by a screening process comprising:

contacting a preparation comprising said expression product or a fragment of said expression product or a genetic sequence that modulates the expression of said allele with a test agent; and

detecting a change in the level and/or functional activity of said expression product or said fragment.

In a further aspect, the invention provides a composition for treating or preventing a fibrotic condition, comprising an agent as broadly described above, together with a pharmaceutically acceptable carrier.

According to a further aspect of the invention there is provided a method for treating or preventing a fibrotic condition, comprising administering to a patient in need of such treatment an effective amount of an agent as broadly described above or an agent-containing composition as broadly described above.

### BRIEF DESCRIPTION OF THE SEVERAL VIEWS OF THE DRAWINGS

FIG. 1 illustrates inheritance of the pro-fibrotic TGF(beta)1 25 Arg/Arg and AT-6 A/A genotypes. The stages of fibrosis (mean±sem) are shown for HCV patients who inherited neither, either or both pro-fibrotic genotypes. * p=0.0019 “neither” compared with “either”; # p=0.0418 “either” compared with “both”.

### DETAILED DESCRIPTION OF THE INVENTION

The present invention relates to an allele of a transforming growth factor (beta) (TGF-(beta)) gene, to an allele of a gene belonging to the same regulatory or biosynthetic pathway as a TGF-(beta) gene such as a gene member of the renin-angiotensin system (RAS) and to expression products of such alleles wherein said alleles or expression products correlate with the development of a-fibrotic condition. The invention further extends to antigen-binding molecules that are immuno-interactive specifically with profibrotic polypeptides encoded by such alleles and to the use of these antigen-binding molecules, the profibrotic polypeptides and polynucleotides encoding them, in assays for diagnosing the presence of, or a predisposition to develop, a fibrotic condition. The invention also encompasses methods for diagnosing the presence of, or a predisposition to develop, a fibrotic condition, particularly hepatic fibrosis, by detecting modulation of the level and/or functional activity of an expression product of an allele of a gene belonging to the same regulatory or biosynthetic pathway as a TGF-(beta) gene. The invention also features a method of screening for agents that modulate the level and/or functional activity of a said expression product and to the use of such modulatory agents in methods for treating and/or preventing a fibrotic condition.

A brief description of each of the sequences disclosed herein is listed in table A.

TABLE A

| SEQ ID NO: | DESCRIPTION | LENGTH | |
|---|---|---|---|
| 1 | Nucleotide sequence corresponding to the coding sequence of | 1821 | nts |
| | a first TGF-(beta)1 allele | | |
| 2 | TGF-(beta)1 polypeptide encoded by SEQ ID NO: 1 | 390 | aa |
| 3 | Nucleotide sequence of promoter region of a first AT allele | 1278 | nts |
| 4 | Nucleotide sequence corresponding to the coding sequence of | 1821 | nts |
| | a second TGF-(beta)1 allele | | |
| 5 | TGF-(beta)1 polypeptide encoded by SEQ ID NO: 4 | 390 | aa |
| 6 | Nucleotide sequence of promoter region of a second AT allele | 1278 | nts |

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

“Allele” is used herein to refer to a variant of a gene found at the same place or locus of a chromosome.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in a mammal to which it is administered, wherein the response includes the production of elements which specifically bind the polypeptide or fragment thereof.

The term “biological sample” as used herein refers to a sample that may be extracted, untreated, treated, diluted or concentrated from a patient. Suitably, the biological sample is selected from tissue samples including tissue from the heart, lungs, kidneys and liver.

By “biologically active fragment” is meant a fragment of a full-length parent polypeptide which fragment retains the activity of the parent polypeptide. A biologically active fragment will therefore have, for example, the activity of TGF-(beta)1 to contribute to the activation of stellate cells and their production of extracellular matrix proteins or the ability to elicit the production of elements that specifically bind to TGF-(beta)1. As used herein, the term “biologically active fragment” includes deletion variants and small peptides, for example of at least 10, preferably at least 20 and more preferably at least 30 contiguous amino acids, which comprise the above activities. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount,” in the context of treating or preventing a condition is meant the administration of that amount of active to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “function” refers to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ non-translated sequences).

“Homology” refers to the percentage number of amino acids that are identical or constitute conservative substitutions as defined in Table 1 below. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Reference herein to “immuno-interactive” includes reference to any interaction, reaction, or other form of association between molecules and in particular where one of the molecules is, or mimics, a component of the immune system.

By “immuno-interactive fragment” is meant a fragment of the polypeptide set forth in any one of SEQ ID NO: 2 and 5 which fragment elicits an immune response, including the production of elements that specifically bind to said polypeptide, or variant or derivative thereof. As used herein, the term “immuno-interactive fragment” includes deletion variants and small peptides, for example of at least six, preferably at least 8 and more preferably at least 20 contiguous amino acids, which comprise antigenic determinants or epitopes. Several such fragments may be joined together.

By “isolated” is meant material that is substantially or essentially free from components that normally accompany it in its native state.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant that transcriptional and translational regulatory polynucleotides are positioned relative to a polypeptide-encoding polynucleotide in such a manner that the polynucleotide is transcribed and the polypeptide is translated.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically acceptable carrier” is meant a solid or liquid filler, diluent or encapsulating substance that can be safely used in topical or systemic administration to a animal, preferably a mammal including humans.

The term “polynucleotide” or “nucleic acid” as used herein designates mRNA, RNA, cRNA, cDNA or DNA. The term typically refers to oligonucleotides greater than 30 nucleotide residues in length.

“Polypeptide,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary,” it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide,” through complementary base pairing. Probes can bind target polynucleotides lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, i.e., through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence,” “comparison window,” “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology,” John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing.

“Stringent conditions” refers to temperature and ionic conditions under which only nucleotide sequences having a high frequency of complementary bases will hybridize. The stringency required is nucleotide sequence dependent and depends upon the various components present during hybridization and subsequent washes, and the time allowed for these processes. Generally, in order to maximize the hybridization rate, non-stringent hybridization conditions are selected; about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridized sequences. Those skilled in the art will recognize that other physical and chemical parameters in the hybridization and wash stages can also be altered to affect the outcome of a detectable hybridization signal from a specific level of homology between target and probe sequences.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxyirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (GENETICIN®) and the hph gene which confers resistance to the antibiotic hygromycin B.

As used herein, underscoring or italicizing the name of a gene shall indicate the gene, in contrast to its protein product, which is indicated in the absence of any underscoring or italicizing. For example, “TGF-(beta)1” shall mean the TGF-(beta)1 gene or transcript thereof, whereas “TGF-(beta)1” shall indicate the protein product of the “TGF-(beta)1” gene.

Description

2. Target Molecules Linked to the Development of a Fibrotic Condition

The present invention is predicated in part on the discovery that TGF-(beta) alleles and/or alleles of a gene belonging to the same regulatory or biosynthetic pathway as a TGF-(beta) gene may cause TGF-(beta) to be produced at a level and/or functional activity that correlates with the development and/or progression of a fibrotic condition.

Thus, the invention features a method for diagnosing the presence of, or a predisposition to develop, a fibrotic condition in a patient, wherein said fibrotic condition is other than lung fibrosis. This method comprises detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) allele, wherein said allele permits the production of a TGF-(beta) polypeptide at a level and/or functional activity that correlates with the development of said condition.

Suitably, the allele is an allele of the TGF-(beta)1 gene. Preferably, the TGF-(beta)1 allele comprises a polymorphism within a signal sequence-encoding portion of the allele. In a preferred embodiment, the TGF-(beta)1 allele encodes an arginine residue at codon 25 relative to the full-length open reading frame of TGF-(beta)1. In this instance, said TGF-(beta)1 allele may comprise, for example, the sequence set forth in SEQ ID NO: 1, which encodes the TGF-(beta)1 polypeptide comprising the sequence set forth in SEQ ID NO: 2. This sequence has been found to correlate with an increased risk of developing a fibrotic condition. Alternatively, the TGF-(beta)1 allele encodes a proline residue at codon 25 relative to the full-length open reading frame of TGF-(beta)1. In this instance, the TGF-(beta)1 allele may comprise, for example, the sequence set forth in SEQ ID NO: 4, which encodes the TGF-(beta)1 polypeptide comprising the sequence set forth in SEQ ID NO: 5. This sequence has been found to correlate with a lower risk of developing a fibrotic condition.

Suitably, said allele of said gene, which belongs to the same regulatory or biosynthetic pathway as the TGF-(beta) gene, is an allele of a gene member of the RAS. In a preferred embodiment of this type, said allele of said gene member permits angiotensin II (AII) to be produced at a level sufficient to induce the production of a TGF-(beta) polypeptide, preferably a TGF-(beta)1 polypeptide, at said level and/or functional activity that correlates with the development of the fibrotic condition. More preferably, the allele is an allele of the angiotensinogen (AT) gene. Suitably, the AT allele comprises a polymorphism within its promoter region. Preferably, said AT allele comprises an adenine nucleotide six bases upstream from the transcription start site of AT. In this instance, said AT allele may comprise, for example, the sequence set forth in SEQ ID NO: 3. This sequence has been found to correlate with an increased risk of developing a fibrotic condition. Alternatively, the AT allele comprises a guanine nucleotide six bases upstream from the transcription start site of AT. In this instance, said AT allele may comprises, for example, the sequence set forth in SEQ ID NO: 6. This sequence has been found to correlate with the absence of, or a lower risk of developing, a fibrotic condition.

The fibrotic condition is suitably selected from cardiac fibrosis, kidney fibrosis, hepatic fibrosis or fibrosis of any other tissue or organ. Preferably, the fibrotic condition is a progressive fibrosis, more preferably progressive hepatic fibrosis.

In a preferred embodiment, the patient is infected with HCV.

It will be understood that the invention contemplates detection of any allele as broadly described above or expression product thereof, which correlates with a risk of developing a fibrotic condition. Such alleles may be obtained from individuals affected with a fibrotic condition.

Nucleic acid isolation protocols are well known to those of skill in the art. For example, an isolated polynucleotide corresponding to gene or allele as broadly described above may be prepared according to the following procedure:

(a) creating primers which flank an allele as broadly described or transcript thereof, above, or a portion of said allele or transcript;

(b) obtaining a nucleic acid extract from an individual affected with a fibrotic condition; and

(c) using said primers to amplify, via nucleic acid amplification techniques, at least one amplification product from said nucleic acid extract, wherein said amplification product corresponds to allele or transcript linked to the development of said condition.

Suitable nucleic acid amplification techniques are well known to the skilled artisan, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-(beta) replicase amplification as for example described by Tyagi et al., (1996, Proc. Natl. Acad. Sci. USA 93: 5395-5400).

The invention also encompasses a method for determining a patient's risk of developing a fibrotic condition other than lung fibrosis. For example, a patient may be diagnosed as having a higher risk of developing said condition by detecting in a biological sample obtained from said patient at least two target molecules selected from different alleles of a TGF-(beta) gene or expression products thereof and different alleles of a gene belonging to the same regulatory or biosynthetic pathway as a TGF-(beta) gene or expression products thereof, wherein each of said different alleles is associated with said higher risk.

The method preferably comprises detecting a pair of target molecules. In one embodiment, the target molecule is a TGF-(beta)1 allele, or transcript thereof, encoding an arginine residue at codon 25 relative to the full-length open reading frame of TGF-(beta)1. In another embodiment, the target molecule is an allele of AT comprising a polymorphism within its promoter region, which polymorphism preferably comprises an adenine nucleotide six bases upstream from the transcription start site of the AT allele. Accordingly, detection of a pair of such target molecules would suggest that the patient is at a higher risk of developing said condition. Preferably, said higher risk is suggested by detecting the presence of said target molecules in a homozygous state.

A patient may be diagnosed as having a lower risk of developing a fibrotic condition other than lung fibrosis by detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein each of said different alleles is associated with said lower risk. In one embodiment, the target molecule is a TGF-(beta)1 allele, or transcript thereof, encoding a proline residue at codon 25 relative to the full-length open reading frame of TGF-(beta)1. In another embodiment, the target molecule is an allele of AT comprising a polymorphism within its promoter region, which polymorphism preferably comprises a guanine nucleotide six bases upstream from the transcription start site of the AT allele. Accordingly, detection of a pair of such target molecules would suggest that the patient is at a lower risk of developing said condition.

A patient may be diagnosed as having an intermediate risk of developing a fibrotic condition other than lung fibrosis by detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein at least one of said different alleles correlates with a higher risk of developing said condition and at least one other of said different alleles correlates with a lower risk of developing said condition. In one embodiment, the method comprises detecting a pair of target molecules, wherein one member of said pair is a TGF-(beta)1 allele, or transcript thereof, encoding a proline residue at codon 25 relative to the full-length open reading frame of TGF-(beta)1, and the other member of said pair is a TGF-(beta)1 allele, or transcript thereof, encoding an arginine residue at codon 25 relative to the full-length open reading frame of TGF-(beta)1. In another embodiment, the method comprises detecting a pair of target molecules, wherein one member of said pair is an allele of AT comprising a guanine nucleotide six bases upstream from the transcription start site of the AT allele and the other member of said pair is an allele of AT comprising an adenine nucleotide six bases upstream from the transcription start site of the AT allele. Accordingly, detection of a pair of such target molecules would suggest that the patient is at an intermediate risk of developing said condition.

The invention also resides in a method for diagnosing the presence of, or a predisposition to develop, a fibrotic condition in a patient, comprising detecting in a biological sample obtained from said patient a level and/or functional activity of an expression product of a TGF-(beta) gene, wherein said level and/or functional activity correlates with the development of said condition.

The invention also extends to a method for diagnosing the presence of, or a predisposition to develop, a fibrotic condition in a patient, comprising detecting in a biological sample obtained from said patient a level and/or functional activity of an expression product of a gene belonging to the same regulatory or biosynthetic pathway as a TGF-(beta) gene, wherein said level and/or functional activity correlates with the development of said condition.

The invention also resides in a method for diagnosing the presence of, or a predisposition to develop, a fibrotic condition in a patient, comprising detecting in a biological sample obtained from said patient a target molecule selected from an allele of a gene belonging to the renin-angiotensin system (RAS), and an expression product of said allele, wherein said allele correlates with the development of said condition.

In a preferred embodiment, said gene is the AT gene.

In an especially preferred embodiment, the allele comprises a guanine nucleotide six bases upstream from its transcription start site.

The invention also encompasses a method for diagnosing the presence of, or a predisposition to develop, a fibrotic condition in a patient, comprising detecting in a biological sample obtained from said patient a level and/or functional activity of an expression product of a gene belonging to the renin-angiotensin system (RAS), wherein said level and/or functional activity is associated with the development of said condition.

3. Vectors

A polynucleotide according to the invention is suitably rendered expressible in a host cell by operably linking the polynucleotide with a regulatory polynucleotide. The synthetic construct or vector thus produced may be introduced firstly into an organism or part thereof before subsequent expression of the construct in a particular cell or tissue type. Any suitable organism is contemplated by the invention, which may include unicellular as well as multi-cellular organisms. Suitable unicellular organisms include bacteria. Exemplary multi-cellular organisms include yeast, mammals and plants.

The construction of the vector may be effected by any suitable technique as for example described in the relevant sections of Ausubel et al. (supra) and Sambrook et al. (supra). However, it should be noted that the present invention is not dependent on and not directed to any one particular technique for constructing the vector.

Regulatory polynucleotides which may be utilized to regulate expression of the polynucleotide include, but are not limited to, a promoter, an enhancer, and a transcriptional terminator. Such regulatory sequences are well known to those of skill in the art. Suitable promoters that may be utilized to induce expression of the polynucleotides of the invention include constitutive promoters and inducible promoters.

4. Antigen-Binding Molecules

The invention also contemplates antigen-binding molecules that bind specifically to the polypeptide encoded by an allele linked to a fibrotic condition or to a fragment of said polypeptide. For example, the antigen-binding molecules may comprise whole polyclonal antibodies. Such antibodies may be prepared, for example, by injecting a polypeptide of the invention or fragment thereof into a production species, which may include mice or rabbits, to obtain polyclonal antisera. Methods of producing polyclonal antibodies are well known to those skilled in the art. Exemplary protocols which may be used are described for example in Coligan et al., Current Protocols In Immunology, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (1991, supra) by immortalizing spleen or other antibody producing cells derived from a production species which has been inoculated with a polypeptide of the invention or a fragment thereof.

The invention also contemplates as antigen-binding molecules Fv, Fab, Fab′ and F(ab′)₂ immunoglobulin fragments. Alternatively, the antigen-binding molecule may comprise a synthetic stabilized Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al. (1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plünckthun et al. (1996, In Antibody engineering: A practical approach. 203-252).

Alternatively, the synthetic stabilized Fv fragment comprises a disulfide stabilized Fv (dsFv) in which cysteine residues are introduced into the V_{H} and V_{L} domains such that in the fully folded Fv molecule the two residues will form a disulfide bond therebetween. Suitable methods of producing dsFv are described for example in (Glockscuther et al. Biochem. 29: 1363-1367; Reiter et al., 1994, J. Biol. Chem. 269: 18327-18331; Reiter et al., 1994, Biochem. 33: 5451-5459; Reiter et al., 1994. Cancer Res. 54: 2714-2718; Webber et al., 1995, Mol. Immunol. 32: 249-258).

Also contemplated as antigen-binding molecules are single variable region domains (termed dAbs) as for example disclosed in (Ward et al., 1989, Nature 341: 544-546; Hamers-Casterman et al., 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomized to create complementarity determining regions (CDRs), which have been selected for antigen binding.

The antigen-binding molecule may be multivalent (i.e., having more than one antigen-binding site). Such multivalent molecules may be specific for one or more antigens. Multivalent molecules of this type may be prepared by dimerization of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerization may be facilitated by fusion of the antibody fragments to amphiphilic helices that naturally dimerize (Pack P. Plünckthun, 1992, Biochem. 31: 1579-1584), or by use of domains (such as the leucine zippers jun and fos) that preferentially heterodimerize (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bi-specific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra).

The antigen-binding molecules can be used to screen expression libraries for variant mutant polypeptides of the invention as described herein. They can also be used to detect polypeptide mutants, polypeptide mutant fragments, variants and derivatives of the invention as described hereinafter.

Methods of detecting modulation of the level of an expression product of a gene or allele linked to a fibrotic condition

Assays for detecting modulation of the level and/or functional activity of a polypeptide encoded by a said gene or allele

Any method of directly or indirectly detecting modulation in the level and/or functional activity of the target molecule is encompassed by the present invention. For example, such detection can be achieved utilizing techniques including, but not restricted to, immunoassays such as Western blotting and ELI SAs, and RT-P CR. For example, in one embodiment, a biological sample from a patient is contacted with an antigen-binding molecule that is specifically immuno-interactive with a polypeptide encoded by an allele, or transcript thereof, linked to a fibrotic condition. The concentration of a complex comprising the polypeptide and the antigen-binding molecule is measured in the contacted sample and the measured complex concentration is then related to the concentration of the polypeptide in the sample. Preferably, the concentration of said polypeptide is compared to a reference or baseline level of said polypeptide corresponding to the presence of, or a high risk of developing, a fibrotic condition. The presence of the fibrotic condition is diagnosed if the concentration of the polypeptide corresponds to the reference level concentration.

It will be appreciated that assays may detect or measure modulation of a genetic sequence from which the target polypeptide of interest is regulated or expressed. In another example, the subject of detection could be a downstream regulatory target of the polypeptide, rather than polypeptide itself.

5.2. Detection of Specific Polypeptides Encoded by a Said Gene or Allele

It is to be understood that although the following discussion is specifically directed to human patients, the teachings are also applicable to any animal that expresses a polypeptide encoded by allele or transcript thereof in accordance with the present invention, such that clinical manifestations such as those seen in patients with a fibrotic condition are found.

It will be appreciated that the methods described herein are applicable to any patient suspected of developing, or having, a said condition, whether such condition is manifest at a young age or at a more advanced age in a patient's life.

The diagnostic and screening methods of the invention are especially useful for a patient suspected of being at risk of developing a said condition based on family history, or a patient in which it is desired to diagnose or eliminate the presence of that condition as a causative agent underlying a patient's symptoms.

5.2.1. Screening for Specific Polypeptides Encoded by a Gene or Allele Linked to a Fibrotic Condition

Screening or diagnosis of a fibrotic condition other than lung fibrosis, or a predisposition to develop such condition, in a patient is now possible by detecting a polypeptide linked to that condition. For example, the presence or absence of a polypeptide linked to a said condition in a patient may determined by isolating a biological sample from a patient, contacting the sample with an antigen-binding molecule as described in Section 4 and detecting the presence of a complex comprising the said antigen-binding molecule and the said polypeptide in said contacted sample.

Any suitable technique for determining formation of the complex may be used. For example, an antigen-binding molecule according to the invention, having a reporter molecule associated therewith may be utilized in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “Current Protocols In Immunology” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043; 4,424,279; and 4,018,653. These include both single-site and two-site assays of the non-competitive types, as well as the traditional competitive binding assays. These assays also include direct binding of a labeled antigen-binding molecule to a target antigen.

Two site assays are particularly favored for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labeled with a reporter molecule capable of producing a detectable signal is then added and incubated, allowing time sufficient for the formation of another complex of antibody-antigen-labeled antibody. Any non-reacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labeled antibody are added simultaneously to the bound antibody. These techniques are well known to those skilled in the art, including minor variations as will be readily apparent. In accordance with the present invention, the sample is one that might contain an antigen including tissue biopsies, serum, whole blood, plasma or lymph fluid.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labeled antibody that binds, as determined by the associated reporter molecule, is proportional to the amount of antigen bound to the immobilized first antibody.

An alternative assay involves immobilizing the antigen in the biological sample and then exposing the immobilized antigen to specific antibody that may or may not be labeled with a reporter molecule. Depending on the amount of target and the strength of the reporter molecule signal, a bound antigen may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthamide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241; U.S. Pat. No. 4,843,000; and U.S. Pat. No. 4,849,338. Suitable enzymes useful in the present invention include alkaline phosphatase, horseradish peroxidase, luciferase, (beta)-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al.), 5,326,692 (Brinkley et al.). Alternatively, reference may be made to the fluorochromes described in U.S. Pat. Nos. 5,227,487; 5,274,113; 5,405,975; 5,433,896; 5,442,045; 5,451,663; 5,453,517; 5,459,276; 5,516,864; 5,648,270; and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable color change. Examples of suitable enzymes include those described supra. It is also possible to employ fluorogenic substrates, which yield a fluorescent product rather than the chromogenic substrates noted above. In all cases, the enzyme-labeled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthamide, europium (EU), may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. The fluorescent-labeled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

5.3. Screening for Polynucleotide Corresponding to a Gene or Allele Linked to a Fibrotic Condition

In another embodiment, the invention provides a method of screening a patient for a polynucleotide linked to a fibrotic condition, comprising isolating a biological sample from the patient, and detecting said polynucleotide by a suitable nucleic acid detection technique.

According to the invention, pre-symptomatic screening of a patient for their likelihood of developing a said condition is now possible by detecting a gene or allele as broadly described above linked to that condition. The screening method of the invention allows a pre-symptomatic diagnosis, including prenatal diagnosis, for the presence of an allele or transcript thereof in such a patient and thus the basis for an opinion concerning the likelihood that such patient would develop or has developed a said condition or symptoms thereof.

For example, in the method of screening, a tissue sample can be taken from a patient, and screened for the presence of one or more alleles associated with a low risk or a high risk of developing a fibrotic condition. Such genes or alleles can be characterized based upon, for example, detection of restriction digestion patterns in ‘normal’ versus the patient's DNA, including Restriction Fragment Length Polymorphism (RFLP) analysis, using nucleic acid probes prepared against those gene or alleles (or fragments thereof). Similarly, mRNA may be characterized and compared to a reference mRNA level and/or size as found in human population not at risk of developing a said condition using similar probes.

Alternatively, a nucleic acid extract from the patient may be utilized in concert with oligonucleotide primers corresponding to sense and antisense sequences of a polynucleotide sequence under test, or flanking sequences thereof, in a nucleic acid amplification reaction such as PCR, or the ligase chain reaction (LCR) as for example described in International Application WO 89/09385. A variety of automated solid-phase detection techniques are also appropriate. For example, very large scale immobilized primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art. Preferably, at least one of said primers is an allele-specific primer specific for the polynucleotide under test. Alternatively, the presence or absence of a restriction endonuclease cleavage site resulting from a mutation in a gene of the present invention may be taken advantage by subjecting a polynucleotide corresponding thereto to digestion with the restriction endonuclease. Accordingly, the present invention encompasses detecting a gene or allele as described herein by RFLP analysis.

Alternatively, the nucleic acid polymorphism in a gene linked to a fibrotic condition may be detected using first-nucleotide change technology described by Dale et al. in U.S. Pat. No. 5,856,092.

The presence in the biological sample of a size pattern (e.g., generated by RFLP), and/or mRNA sizes or levels and/or a gene or allele linked to a fibrotic condition would indicate that the patient has developed or is at risk of developing a symptom associated with a fibrotic condition.

Prenatal diagnosis can be performed when desired, using any known method to obtain fetal cells, including amniocentesis, chorionic villous sampling (CVS), and fetoscopy. Prenatal chromosome analysis can be used to determine if the portion of the respective chromosomes possessing one or more gene or alleles described herein are present in a heterozygous state.

Detection Kits

The present invention also provides kits for the detection in a biological sample of a gene or allele linked to a fibrotic condition, or a polypeptide encoded thereby. These will contain one or more particular agents described above depending upon the nature of the test method employed. In this regard, the kits may include one or more of a polypeptide, antigen-binding molecule and polynucleotide according to the invention. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention, and optionally a DNA polymerase, DNA ligase etc depending on the nucleic acid amplification technique employed.

Identification of Target Molecule Modulators

The invention also features a method of screening for an agent that modulates the level and/or functional activity of an expression product of an allele selected from an allele of a TGF-(beta) gene and an allele of a gene belonging to the same regulatory or biosynthetic pathway as a TGF-(beta) allele. The method comprises contacting a preparation comprising an expression product as broadly described above or fragment thereof with a test agent and detecting a change in the level and/or functional activity of said expression product or said fragment.

Screening for modulatory agents according to the invention can be achieved by any suitable method. For example, the method may include contacting a cell comprising a polynucleotide corresponding to an of allele a TGF-(beta) gene or of a gene belonging to the same regulatory or biosynthetic pathway as a TGF-(beta) gene, with an agent suspected of having said modulatory activity and screening for the modulation of the level and/or functional activity of a protein encoded by said polynucleotide, or the modulation of the level of an expression product encoded by the polynucleotide, or the modulation of the activity or expression of a downstream cellular target of said protein or said expression product. Detecting such modulation can be achieved utilizing techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g., a domain such as a protein binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells, immortalized cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, (beta)-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in said level from an established baseline may be detected using any of the techniques described above and well known in the art. In particularly preferred embodiments, the cells are epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, (beta)-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modeling, and other routine procedures employed in rational drug design.

In another embodiment, a method of identifying agents that inhibit the activity of a polypeptide encoded by an allele of a TGF-(beta) gene or of a gene belonging to the same regulatory or biosynthetic pathway as TGF-(beta) is provided in which a purified preparation of the polypeptide in the presence and absence of a candidate agent under conditions in which the polypeptide is active, and the level of activity of the polypeptide is measured by a suitable assay. For example, a polypeptide inhibitor can be identified by measuring the ability of a candidate agent to decrease polypeptide activity in a cell (e.g., a hepatic cell, a cardiac cell, a kidney cell). In this method, a cell that is capable of expressing a polynucleotide corresponding to an allele of a TGF-(beta) gene or of a gene belonging to the same regulatory or biosynthetic pathway as a TGF-(beta) allele, is exposed to, or cultured in the presence and absence of, the candidate agent under conditions in which the polynucleotide-encoded polypeptide is active in the cell, and an activity such as stellate cell activation and/or production of extracellular matrix proteins is detected. An agent tests positive if it inhibits any of these activities.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesized by any suitable technique. Such molecules may be conveniently prepared by a person skilled in the art using standard protocols as for example described in Sambrook, et al., Molecular Cloning: A Laboratory Manual (Cold Spring Harbor Press, 1989), in particular Sections 16 and 17; Ausubel et al., Current Protocols In Molecular Biology (John Wiley & Sons, Inc. 1994-1998), in particular Chapters 10 and 16; and Coligan et al., Current Protocols In Protein Science (John Wiley & Sons, Inc. 1995-1997), in particular Chapters 1, 5 and 6. Alternatively, a target polypeptide according to the invention may be synthesized using solution synthesis or solid phase synthesis as described, for example, in Chapter 9 of Atherton and Shephard (supra) and in Roberge et al. (1995, Science 269: 202).

To identify and isolate the peptide/solid phase support that interacts and forms a complex with a target molecule, preferably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothiocyanate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labeling with enzymes, fluorescent dyes or colored or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a Petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes color, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labeled epitope specific antigen-binding molecule. Once isolated, the identity of the peptide attached to the solid phase support may be determined by peptide sequencing.

Therapeutic and Prophylactic Uses

A further feature of the invention is the use of a modulatory agent according to Section 7 as actives (“therapeutic agents”) in pharmaceutical compositions for treatment or prophylaxis of a fibrotic condition. Examples of such modulatory agents include, but are not limited to, inhibitors of angiotensin I-converting enzyme (ACE) and antagonists of angiotensin II (AII) receptor.

Preferably, but not exclusively, the ACE inhibitors are selected from any one or more of enalapril maleate, quinapril hydrochloride, captopril, perindopril erbumine, trandolapril, fosinopril sodium and ramipril.

Preferably, but not exclusively, the AII receptor antagonists are selected from any one or more of candesartan cilexetil, irbesartan, losartan and telmisartan

Thus, the invention extends to a method for treating or preventing the development of a fibrotic condition, comprising administering to a patient in need of such treatment an effective amount of a modulatory agent as broadly described above. In a preferred embodiment, the fibrotic condition is selected from cardiac fibrosis, kidney fibrosis, hepatic fibrosis or fibrosis of any other tissue or organ. Preferably, the fibrotic condition is a progressive fibrosis, more preferably progressive hepatic fibrosis. The fibrotic condition is suitably selected from cardiac fibrosis, kidney fibrosis, lung fibrosis, hepatic fibrosis or fibrosis of any other tissue or organ. Preferably, the fibrotic condition is hepatic fibrosis, more preferably progressive hepatic fibrosis.

A pharmaceutical composition according to the invention is administered to a patient, preferably prior to such symptomatic state associated with the condition(s). The therapeutic agent present in the composition is provided for a time and in a quantity sufficient to treat that patient.

Suitably, the pharmaceutical composition comprises a pharmaceutically acceptable carrier. Depending upon the particular route of administration, a variety of pharmaceutically acceptable carriers, well known in the art may be used. These carriers may be selected from a group including sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the immunogenic agents of the invention with liquid carriers or finely divided solid carriers or both, and then, if necessary, shaping the product into the desired presentation.

The above compositions may be administered in a manner compatible with the dosage formulation, and in such amount as is therapeutically-effective to alleviate patients from symptoms related to the condition(s), or in amounts sufficient to protect patients from developing symptoms related to the condition(s). The dose administered to a patient, in the context of the present invention, should be sufficient to effect a beneficial response in a patient over time such as the therapeutic or prophylactic effects mentioned above. The quantity of the therapeutic agent(s) to be administered may depend on the subject to be treated inclusive of the age, sex, weight and general health condition thereof. In this regard, precise amounts of the therapeutic agent(s) for administration will depend on the judgement of the practitioner. In determining the effective amount of the therapeutic agent to be administered in the treatment of, or prophylaxis against, the condition(s), the physician may evaluate progression of the condition(s). In any event, suitable dosages of the therapeutic agents of the invention may be readily determined by those of skill in the art. Such dosages may be in the order of nanograms to milligrams of the therapeutic agents of the invention.

The invention further contemplates cells or tissues containing therein a vector of the invention, or alternatively, cells or tissues produced from the treatment method of the invention.

In order that the invention may be readily understood and put into practical effect, particular preferred embodiments will now be described by way of the following non-limiting examples.

### EXAMPLES

### Example 1

Patients

The present study involved 128 consecutive Caucasian patients with chronic HCV who had undergone liver biopsy at the Princess Alexandra Hospital, Brisbane between February 1995 and May 1999 and for whom genomic DNA was available. Patients of ethnicity other than Caucasian were excluded from the study in order to provide a homogeneous group for valid comparisons. Informed consent was obtained from each patient and the Princess Alexandra Hospital Research Ethics Committee approved the study protocol. Diagnosis of chronic HCV was based on standard serological assays and abnormal serum aminotransferase levels (greater or equal to 1.5× upper limit of normal) for at least 6 months. All patients were positive for HCV antibody by the second-generation ELISA (Abbott Labs, North Chicago, Ill., USA) with infection confirmed by detection of circulating HCV RNA by polymerase chain reaction (PCR) using the AMPLICOR™ HCV assay (Roche, N.J., USA) and were negative for HBsAg or antibodies to human immunodeficiency virus. Viral genotyping was performed in 80 patients using the Inno-Lipa HCV II assay (Innogenetics, Zwijnaarde, Belgium). None of the patients were receiving ACE-inhibitors or angiotensin II receptor antagonists. Patients with other forms of chronic liver disease were excluded from the analysis.

Details about weight, height and average alcohol intake (g/day) during the preceding 12 months were obtained from all patients at the time of liver biopsy (current alcohol intake). Information regarding average alcohol intake (g/day) prior to the last 12 months was also obtained (past alcohol intake). Alcohol consumption was assessed retrospectively by interview on at least 3 occasions. The number and types of alcoholic drinks consumed each day were recorded and the alcohol content of each drink was calculated. The alcohol intake over a weekly period was averaged and recorded in grams per day.

A single pathologist (AC) assessed all biopsies, which were taken prior to treatment with antiviral therapy, without knowledge of patients' clinical or laboratory data. Liver biopsy specimens were fixed in buffered formalin and embedded in paraffin. The degree of inflammation and fibrosis was assessed and graded according to the method of Scheuer (16) and steatosis was graded as described previously (2). Perls' stain was available in 59 patients and was graded 0 to 4.

### Example 2

Polymorphism Genotyping

Genomic DNA was extracted from peripheral blood mononuclear cells (PBMC) using the Progenome II DNA isolation kit (Progen, Brisbane, Australia) or DNAzol™ Reagent (Life Technologies, Australia). The bi-allelic polymorphisms for the IL-10 (17), TGF-(beta)1 (18), and ACE (19) genes were detected using polymerase chain reaction-based methods as previously described. Qiagen HOTSTAR™ Taq polymerase (Qiagen Pty Ltd, Clifton Hill, Victoria, Australia) was used for all genotyping. The TNF(alpha) −308 G/A polymorphism was determined using sequence specific primers. For each sample, duplicate reactions were performed with each reaction containing a common reverse primer, 5′ TCC TCC CTG CTC CGA TTC CG 3′, and a forward primer to amplify either the “A” allele, 5′ CAA TAG GTT TTG AGG GGC ATG A 3′, or the “G” allele, 5′ CAA TAG GTT TTG AGG GGC ATG G 3′. As a positive control each reaction also contained primers designed to amplify exon 2 of the DR(beta)1 gene; 5′ CCC CAC AGC ACG TTT CTT G 3′ and 5′ CCG CTG CAC TGT GAA GCT CT 3′. The AT-6 G/A polymorphism was detected using a PCR-RFLP technique. The primer sequences were 5′ CTC AGT TAC ATC CTG AGA GAG ACA AGA CC 3′ and 5′ GTG TCG CTT CTG GCA TCT GTC CTT CTG G 3′. Following amplification, the PCR products were digested at 60° C. for one hour with BsiE 1 (New England Biolabs, Genesearch Pty Ltd, Arundel, Queensland, Australia), which cuts the product when the G allele is present. All samples were amplified and digested in parallel with 3 samples of known genotype and water.

### Example 3

Statistical Analysis

The allele distribution in patient groups was compared with that of previously published Caucasian control populations using a Chi-square Goodness of Fit test. The influence of cytokine genotypes on the stage of fibrosis was tested using the Mantle-Haenszel chi-squared test for a linear association (20). Multiple ordinal logistic regression analysis was used to assess the influence of cytokine and RAS genotypes on the stage of fibrosis, after adjusting for potential confounding by age, gender, past alcohol intake, portal inflammation, and steatosis. Odds ratios (with 95% confidence intervals) were calculated to estimate the relative risk of increasing stage of liver fibrosis associated with each polymorphism. Bonferroni-type adjustments were not performed and thus the p-values reported throughout have not been corrected for multiple comparisons.

Current alcohol consumption was grouped based on average grams per day (g/d) into either less than or equal to 10 g/d or greater than 10 g/d; past alcohol consumption was also classified as either less than or equal to 50 g/d or greater than 50 g/d. For statistical analyses, patients with advanced fibrosis (stages 3 and 4) were combined.

### Example 4

Polymorphisms of the Cytokine and RAS Genes in Patients with Chronic HCV

The frequencies of the alleles of the TGF-(beta)l, IL-10, TNF-(alpha), angiotensinogen and ACE genes in our Caucasian patients with HCV are summarized in Table 1. With the exception of TNF(alpha)-308, no significant differences in the allele or haplotype frequencies could be demonstrated between the HCV patients and Caucasian control populations derived from previously published studies (9,14,18,21-23). For the TNF(alpha)-308 polymorphism, genotype frequencies in Control populations are inconsistent between studies. When compared with the frequencies determined by Perrey et al. (21) and Tanaka et al. (24), there appears to be an under-representation and over-representation respectively of the “AA” genotype in our HCV patients. However, there was no significant difference when the frequency of this polymorphism in our HCV patients was compared with the Control populations in studies by Huang et al. (25) and Czaja et al. (26).

Table 1 depicts cytokine and ras gene polymorphism frequencies in hcv patients

TABLE 1

| Polymorphism | Genotype | Phenotype | Frequency |
|---|---|---|---|
| TGF(beta)1 | | | |
| −509 C/T | C/C | uncertain | .55 |
| | C/T | uncertain | .36 |
| | T/T | uncertain | .09 |
| Codon 10 | Leu/Leu | uncertain | .35 |
| | Leu/Pro | uncertain | .46 |
| | Pro/Pro | uncertain | .18 |
| Codon 25 | Arg/Arg | high | .86 |
| | Arg/Pro | intermediate | .12 |
| | Pro/Pro | low | .02 |
| TNF(alpha) | | | |
| −308 | A/A | high | .06 |
| | G/A | high | .22 |
| | G/G | low | .72 |
| IL-10 | | | |
| −1082 | G/G | high | .22 |
| | G/A | intermediate | .52 |
| | A/A | low | .26 |
| −592 | C/C | uncertain | .65 |
| | A/C | uncertain | .34 |
| | A/A | uncertain | .02 |
| ACE | | | |
| intron 16 | D/D | high | .27 |
| | D/I | intermediate | .48 |
| | I/I | low | .25 |
| AT | | | |
| −6 | A/A | high | .18 |
| | G/A | intermediate | .50 |
| | G/G | low | .32 |

The age of the patients (70% male) ranged from 23 to 66 years, with a mean age of 37.9 (SD 6.6) years. The possible source of hepatitis was previous intravenous drug use in 88 (68.8%), post-transfusion in 20 (15.6%) and other risk factors or unknown in 20 (15.6%). Alcohol intake in the past and in the 12 months preceding the liver biopsy ranged from none to very heavy (>540 g/d). The Perl's stain was 0 in 49 patients and 1 in 10 patients. HCV genotype was 1a or 1b in 47 patients, 3a in 30 patients and “other” in 3 patients.

### Example 5

Factors Associated with the Severity of Fibrosis

The stage of fibrosis was 0 in 30 (23.4%), 1 in 44 (34.4%), 2 in 27 (21.1%) and 3 or 4 in 27 (21.1%). A statistically significant relationship was seen between the TGF-(beta)1 gene polymorphism at codon 25 and the stage of hepatic fibrosis (p=0.023) (Table 2). Individuals with the arginine/arginine homozygous genotype were more likely to have increased hepatic fibrosis compared with individuals inheriting the arginine/proline or the proline/proline homozygous genotype. After adjustment for potential confounders (age, gender, past alcohol consumption, portal inflammation and steatosis), the arginine/arginine genotype (codon 25) remained associated with more severe fibrosis (p=0.018).

TABLE 2

| | | | | | | | Adjusted^{#} |
|---|---|---|---|---|---|---|---|
| | Stage of Fibrosis | | | | Crude | Adjusted^{#} | Odds Ratio |
| Polymorphism | 0 | 1 | 2 | 3/4 | ⁺p-value | ⁺p-value | (95% CI) |
| TGF-509: | n = 29 | n = 43 | n = 27 | n = 27 | | | |
| C/C (n = 59) | *23.7% | 33.9% | 23.7% | 18.7% | | | 1.0 |
| C/T or T/T (n = 67) | 22.4% | 34.3% | 19.4% | 23.9% | 0.69 | 0.35 | 1.41 |
| | | | | | | | (0.69, 2.88) |
| TGF codon 10: | n = 29 | n = 43 | n = 27 | n = 27 | | | |
| Leu/Leu (n = 45) | 24.4% | 31.1% | 28.9% | 15.6% | | | 1.0 |
| Leu/Pro or Pro/Pro | 22.2% | 35.8% | 17.3% | 24.7% | 0.65 | 0.37 | 1.40 |
| (n = 81) | | | | | | | (0.67, 2.93) |
| TGF codon 25: | n = 28 | n = 43 | n = 26 | n = 27 | | | |
| Arg/Arg (n = 106) | 19.8% | 33.0% | 23.6% | 23.6% | | | 1.0 |
| Arg/Pro or Pro/Pro | 38.9% | 44.4% | 5.6% | 11.1% | 0.023 | 0.018 | 0.25 |
| (n = 18) | | | | | | | (0.08, 0.79) |
| TNF-308: | n = 29 | n = 43 | n = 26 | n = 26 | | | |
| G/G (n = 91) | 22.0% | 37.3% | 20.9% | 19.8% | | | 1.0 |
| G/A or A/A (n = 33) | 27.3% | 27.3% | 21.2% | 24.2% | 0.86 | 0.39 | 1.42 |
| | | | | | | | (0.64, 3.15) |
| IL-10-1082: | n = 30 | n = 42 | n = 27 | n = 27 | | | |
| A/A (n = 32) | 28.1% | 21.9% | 28.1% | 21.9% | | | 1.0 |
| G/A or G/G (n = 94) | 22.3% | 37.3% | 19.1% | 21.3% | 0.86 | 0.39 | 0.88 |
| | | | | | | | (0.4, 1.93) |
| IL-10-592: | n = 30 | n = 42 | n = 27 | n = 28 | | | |
| C/C (n = 82) | 18.3% | 36.6% | 22.0% | 23.2% | | | 1.0 |
| C/A or A/A (n = 45) | 33.3% | 26.7% | 20.0% | 20.0% | 0.40 | 0.72 | 1.14 |
| | | | | | | | (0.55, 2.38) |
| ACE: intron 16 | n = 29 | n = 41 | n = 27 | n = 27 | | | |
| I/I (n = 30) | 16.7% | 43.3% | 20.0% | 20.0% | | | 1.0 |
| D/I or D/D (n = 94) | 25.6% | 29.8% | 22.3% | 22.3% | 0.94 | 0.52 | 0.76 |
| | | | | | | | (0.33, 1.75) |
| AT-6: | n = 30 | n = 44 | n = 27 | n = 27 | | | |
| A/A (n = 22) | 18.2% | 18.2% | 18.2% | 45.4% | | | 1.0 |
| G/A or G/G (n = 106) | 24.5% | 37.8% | 21.7% | 16.0% | 0.014 | 0.030 | 0.34 |
| | | | | | | | (0.12, 0.90) |

*All percentages are row percentages
⁺p-values not corrected for multiple comparisons (Bonferroni correction)
^{#}Adjusted for age, gender, steatosis, portal inflammation and past alcohol intake. 95% confidence intervals are given in parentheses.

A statistically significant relationship was also seen between the polymorphism in the promoter region of the angiotensinogen gene (AT-6) and the stage of hepatic fibrosis (p=0.014) (Table 2). Individuals with the adenine/adenine homozygous genotype were more likely to have increased hepatic fibrosis compared with individuals inheriting the adenine/guanine or the guanine/guanine homozygous genotype. This relationship remained significant (p=0.03) after adjustment for potential confounders.

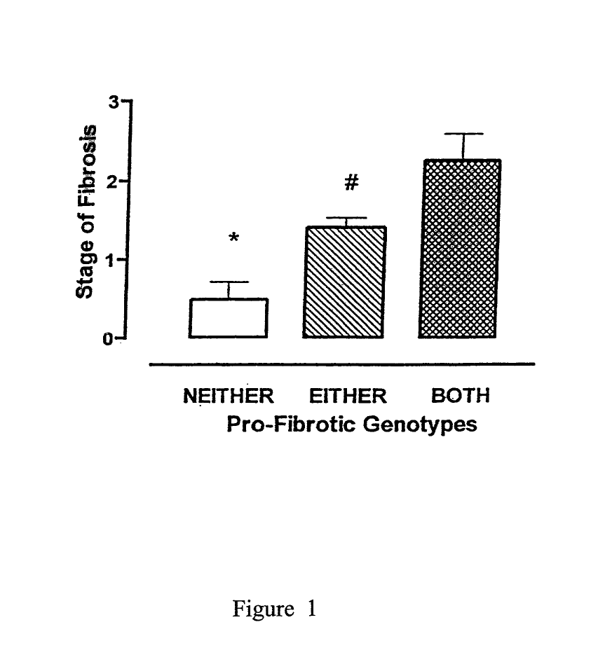

When inheritance of the TGF-(beta)1 and angiotensinogen genotypes was considered together, an even more striking association with hepatic fibrosis was seen (FIG. 1). The patients who inherited neither of the pro-fibrotic genotypes (TGF-(beta)1 codon 25 Arg/Arg or AT-6 A/A) had no or only minimal fibrosis. When comparing the levels of fibrosis for those patients who inherited neither with those who inherited either pro-fibrotic genotype, the p value was 0.0019 with an odds ratio of 0.080 (0.016,0.394) (following multiple logistic regression). Similarly when comparing the levels of fibrosis in those individuals that inherited either pro-fibrotic genotype with those that inherited both pro-fibrotic genotypes, the p value was 0.0418 with an odds ratio 0.330 (0.114, 0.96).

There were no significant relationships between the other TGF-(beta)1 polymorphisms, or the ACE, IL-10 and TNF-(alpha) polymorphisms and the stage of hepatic fibrosis (Table 2). No significant association was found between viral genotype and stage of fibrosis (data not shown).

In view of the above, the present inventors have demonstrated for the first time that host genetic factors may account for some of the variability in the rate of disease progression seen in patients with chronic hepatitis C. A noteworthy relationship has been demonstrated between TGF-(beta)1 and angiotensinogen genotypes and the development of progressive hepatic fibrosis. This association persisted after correcting for potential confounding variables (age, gender, alcohol consumption, portal inflammation and steatosis) that may have independent effects on histological severity.

The TGF-(beta)1 gene polymorphism at codon 25 is significantly associated with TGF-(beta)1 production in vitro (8). Following in vitro stimulation of peripheral blood mononuclear cells, individuals with the arginine/arginine homozygous genotype produce substantially more TGF-(beta)1 protein than individuals with the arginine/proline genotype. In the patient population of the present study, individuals with the high TGF-(beta)1-producing genotype (arginine/arginine) at codon 25 were more likely to have increased hepatic fibrosis compared with individuals inheriting the low TGF-(beta)1-producing arginine/proline or proline/proline genotypes. This polymorphism occurs within the peptide signal sequence that is cleaved from the active TGF-(beta)1 protein (27). The proline/arginine substitution corresponds to an exchange of a small, neutral residue for a charged residue that may have a direct effect on the adjacent cleavage site at codon 29 (8). The regulation of TGF-(beta)1 secretion and activity involves complex post-transcriptional events, including mRNA stabilization, the assembly and activation of the latent TGF-(beta)1 complex, and the modulation of receptor expression (28). It is therefore interesting that genotype differences are associated with variation in the production of TGF-(beta)1.

The functional significance of the other two polymorphic sites within the TGF-(beta)1 gene remains uncertain, and in the patient population investigated they were not associated with the stage of hepatic fibrosis. Similarly, inheritance of particular IL-10 and TNF-(alpha) genotypes did not influence the severity of the liver disease. These results support the dominant role of TGF-(beta)1 in hepatic fibrosis.

The documentation of a relationship between angiotensinogen genotype and fibrosis raises the novel suggestion that AII may be another mediator of extracellular matrix production in the liver. The polymorphism within the promoter region of angiotensinogen has been shown to affect the basal transcription rate of the gene (14). Not wishing to be bound to any one particular theory or mode of operation, it is considered that a modest increase in basal expression of angiotensinogen may lead to chronic elevation in baseline AII production either by the circulating RAS or by tissue RAS.

The present findings raise the possibility that polymorphisms in the TGF-(beta)1 and angiotensinogen genes have a role in determining the progression of fibrosis in chronic HCV. Patients who are genetically predisposed to produce greater amounts of free TGF-(beta)1 protein might be more prone to hepatic fibrosis due to the net increased concentration of this fibrogenic cytokine. Knowledge of these polymorphisms may have prognostic significance in patients with chronic HCV and may direct more aggressive therapy towards those patients with an increased risk of disease progression.

### BIBLIOGRAPHY

The references cited in this section are referred to herein by parenthesized numbers.

Poynard T, Bedossa P, Opolon P. Natural history of liver fibrosis progression in patients with chronic hepatitis C. The OBSVIRC, METAVIR, CLINIVIR, and DOSVIRC groups. Lancet 1997;349(9055):825-32.

Hourigan L F, Macdonald G A, Purdie D, Whitehall V H, Shorthouse C, Clouston A, Powell E E. Fibrosis in chronic hepatitis C correlates significantly with body mass index and steatosis. Hepatology 1999;29:1215-9.

Friedman S L. Cytokines and fibrogenesis. Semin Liver Dis 1999;19:129-40.

Bedossa P, Paradis V. Transforming growth factor-beta (TGF-beta): a key-role in liver fibrogenesis. J Hepatol 1995;22(Suppl):37-42.

Castilla A, Prieto J, Fausto N. Transforming growth factors beta 1 and alpha in chronic liver disease. Effects of interferon alfa therapy. N. Engl. J. Med. 1991;324:933-40.

Wang S C, Ohata M, Schrum L, Rippe R A, Tsukamoto H. Expression of interleukin-10 by in vitro and in vivo activated hepatic stellate cells. J. Biol. Chem. 1998;273:302-8.

Baker S J, Reddy E P. Transducers of life and death: TNF receptor superfamily and associated proteins. Oncogene 1996;12:1-9.

Awad M R, El-Gamel A, Hasleton P, Turner D M, Sinnott P J, Hutchinson I V. Genotypic variation in the transforming growth factor-betal gene: association with transforming growth factor-betal production, fibrotic lung disease, and graft fibrosis after lung transplantation. Transplantation 1998;66: 1014-20.

Turner D M, Williams D M, Sankaran D, Lazarus M, Sinnott P J, Hutchinson I V. An investigation of polymorphism in the interleukin-10 gene promoter. Eur. J. Immunogenet. 1997;24:1-8.

Wilson A G, di Giovine F S, Blakemore A I, Duff G W. Single base polymorphism in the human tumor necrosis factor alpha (TNF alpha) gene detectable by NcoI restriction of PCR product. Hum. Mol. Genet. 1992;1:353.

Kim S K, Ohta K, Hamaguichi A, Omura T, Yukimura T, Miura K, Inada Y et al. Angiotensin II type I receptor antagonist inhibits the gene expression of transforming growth factor-beta 1 and extracellular matrix in cardiac and vascular tissues of hypertensive rats. J. Pharm. Exp. Ther. 1995;273:509-515.

Lee L K, Meyer T W, Pollock A S, Lovett D H. Endothelial cell injury initiates glomerular sclerosis in the rat remnant kidney. J. Clin. Invest. 1995;96:953-964.

Noble N A, Border W A. Angiotensin II in renal fibrosis: Should TGF-(beta) rather than blood pressure be the therapeutic target? Seminars in Nephrology 1997;17:455-466.

Inoue I, Nakajima T, Williams CS, Quackenbush J, Puryear R, Powers M, Cheng T et al. A nucleotide substitution in the promoter of human angiotensinogen is associated with essential hypertension and affects basal transcription in vitro. J. Clin. Invest. 1997;99:1786-1797.

Rigat B, Hubert C, Alhenc-Gelas F, Cambien F, Corvol P, Soubrier F. An insertion/deletionpolymorphism in the angiotensin 1 converting enzyme gene accounting for half of the variance of serum enzyme levels. J Clin Invest 1990;86:1343-1346.

Scheuer P J. Classification of chronic viral hepatitis: a need for reassessment. J. Hepatol. 1991;13:372-74.

Edwards-Smith C J, Jonsson J R, Purdie D M, Bansal A, Shorthouse C, Powell E E. Interleukin-10 promoter polymorphism predicts initial response of chronic hepatitis C to interferon alfa. Hepatology 1999;30:526-30.

Lympany P A, Avila J J, Mullighan C, Marshall S, Welsh K I, du Bois R M. Rapid genotyping of transforming growth factor (beta)1 gene polymorphisms in a UK Caucasoid control population using the polymerase chain reaction and sequence specific primers. Tissue Antigens 1998;52:573-578.

Lindpaintner K, Pfeffer M A, Kreutz R, Stampfer M J, Grodstein F, LaMotte F, Buring J et al. A prospective evaluation of an angiotensin-converting-enzyme gene polymorphism and the risk of ischemic heart disease. N. Eng. J. Med. 1995;332: 706-711.

Mantel N, Haenszel, W. Statistical aspects of the analysis of data from retrospective studies of disease. J. Nat. Cancer Inst. 1959 22:719-48.

Perrey C, Pravica V, Sinnott P J, Hutchinson I V. Genotyping for polymorphisms in interferon-gamma, interleukin-10, transforming growth factor-beta 1 and tumour necrosis factor-alpha genes: a technical report. Transpl. Immunol. 1998;6:193-7.

Huang D, Zhou Y, Xia S, Liu L, Pirskanen R, Lefvert A K. Markers in the promoter region of interleukin-10 (IL-10) gene in myasthenia gravis: implications of diverse effects of IL-10 in the pathogenesis of the disease. J. Neuroimmunol. 1999;94:82-87.

Gayagay G, Yu B, Hambly B, Boston T, Hahn A, Celermajer D S, Trent R J. Elite endurance athletes and the ACE I allele—the role of genes in athletic performance. Human Genetics 1998;103: 48-50.

Tanaka A, Quaranta S, Mattalia A, Coppel R, Rosina F, Manns M, Gershwin M E. The tumor necrosis factor-alpha promoter correlates with progression of primary biliary cirrhosis. J. Hepatol. 1999;30: 826-829.

Huang D R, Pirskanen R, Matell G, Lefvert A K. Tumour necrosis factor-alpha polymorphism and secretion in myasthenia gravis. J. Neuroimmunol. 1999;94:165-171.

Czaja A J, Cookson S, Constantini P M, Clare M, Underhill J A, Donaldson PT. Cytokine polymorphisms associated with clinical features and treatment outcome in type 1 autoimmune hepatitis. Gastroenterology 1999;117: 645-652.

Miyazono K, Hellman U, Wemstedt C, Heldin C H. Latent high molecular weight complex of transforming growth factor beta 1. Purification from human platelets and structural characterization. J. Biol. Chem. 1988;263:6407-15

Kim S J, Park K, Koeller D, Kim K Y, Wakefield L M, Sporn M B, Roberts A B. Post-transcriptional regulation of the human transforming growth factor-beta 1 gene. J. Biol. Chem. 1992;267:13702-7.

Li B, Khanna A, Sharma V, Singh T, Suthanthiran M, August P. TGF-betal DNA polymorphisms, protein levels, and blood pressure. Hypertension 1999;33:271-5.

Cambien F, Ricard S, Troesch A, Mallet C, Generenaz L, Evans A, Arveiler D et al. Polymorphisms of the transforming growth factor-beta 1 gene in relation to myocardial infarction and blood pressure. The Etude Cas-Temoin de l'Infarctus du Myocarde (ECTIM) Study. Hypertension 1996;28:881-7.

Nakatsukasa H, Nagy P, Evarts R P, Hsia C C, Marsden E, Thorgeirsson S S. Cellular distribution of transforming growth factor-beta 1 and procollagen types I, III, and IV transcripts in carbon tetrachloride-induced rat liver fibrosis. J. Clin. Invest. 1990;85:1833-43.

Nakatsukasa H, Evarts R P, Hsia C C, Thorgeirsson S S. Transforming growth factor-beta 1 and type I procollagen transcripts during regeneration and early fibrosis of rat liver. Lab. Invest. 1990;63:171-80.

Hahn A W, Resink T J, Bernhardt J, Ferracin F, Buhler F R. Stimulation of autocrine platelet-derived growth factor AA-homodimer and transforming growth factor (beta) in vascular smooth muscle cells. Biochem. Biophys. Res. Commun. 1991;178:1451-1458.

Gibbons G H, Pratt R E Dzau V J. Vascular smooth muscle cell hypertrophy vs hyperplasia: Autocrine transforming growth factor-(beta)1 expression determines growth responses to angiotensin II. J. Clin. Invest. 1992;90:456-461.

Bataller R, Gines P, Gorbig M N, Nicolas J M, Garcia-Ramallo E, Arroyo V, Rodes J. Angiotensin II induces contraction and is mitogenic for cultured human hepatic stellate cells. Antagonization by losartan and modulation by vasodilators. Hepatology 1998;28:437A.

Rothman K J. No adjustments are needed for multiple comparisons. Epidemiology 1990;1:43-46.

Pemeger T V. What's wrong with Bonferroni adjustments BMJ 1998;316:1236-8

Savitz D A, and Olshan A F. Multiple comparisons and related issues in the interpretation of epidemiologic data. Am. J. Epidemiol. 1995; 142:904-8.

In this disclosure, the designation “(alpha)” is used in place of the Greek letter alpha, and the designation “(beta)” is used in place of the Greek letter beta.

The disclosure of every patent, patent application, and publication cited herein is hereby incorporated herein by reference in its entirety.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

We claim:

1. A method for diagnosing the presence of, or a predisposition to develop, a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, and wherein said allele permits the production of a TGF-(beta) polypeptide at a level and/or functional activity that correlates with the development of said condition.

2. The method of claim 1, wherein said allele is a TGF-(beta)1 allele.

3. The method of claim 2, wherein said TGF-(beta)1 allele comprises a polymorphism within a signal sequence-encoding portion of the allele.

4. The method of claim 2, wherein said TGF-(beta)1 allele encodes an arginine residue at codon 25 relative to the full-length open reading frame of TGF-(beta)1.

5. The method of claim 2, wherein the expression product of said TGF-(beta)1 allele is a polypeptide comprising the sequence set forth in SEQ ID NO: 2.

6. The method of claim 1, wherein said allele of said gene, which belongs to the same regulatory or biosynthetic pathway as the TGF-(beta) gene, is an allele of a gene member of the renin-angiotensin system (RAS).

7. The method of claim 6, wherein said allele permits angiotensin II (AII) to be produced at a level sufficient to induce the production of TGF-(beta)1 at a level and/or functional activity that correlates with the development of said condition.

8. The method of claim 6, wherein said allele is an angiotensinogen (AT) allele.

9. The method of claim 8, wherein said AT allele comprises a polymorphism within its promoter region.

10. The method of claim 8, wherein said AT allele comprises an adenine nucleotide six bases upstream from the transcription start site of AT.

11. The method of claim 8, wherein said AT allele comprises the sequence set forth in SEQ ID NO: 3.

12. The method of claim 1, wherein the fibrotic condition is selected from the group consisting of cardiac fibrosis, kidney fibrosis and hepatic fibrosis.

13. The method of claim 1, wherein the fibrotic condition is a progressive fibrosis

14. The method of claim 1, wherein the fibrotic condition is progressive hepatic fibrosis.

15. The method of claim 1, wherein the patient is infected with chronic hepatitis C virus.

16. A method for diagnosing a higher risk of developing a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, wherein said allele correlates with said higher risk.

17. The method of claim 16, wherein said allele is a TGF-(beta)1 allele.

18. The method of claim 17, wherein said TGF-(beta)1 allele comprises a polymorphism within a signal sequence-encoding portion of the allele.

19. The method of claim 17, wherein said TGF-(beta)1 allele encodes an arginine residue at codon 25 relative to the full-length open reading frame of TGF-(beta)1.

20. The method of claim 17, wherein the expression product of said TGF-(beta)1 allele is a polypeptide comprising the sequence set forth in SEQ ID NO: 2.

21. The method of claim 16, wherein said allele of said gene, which belongs to the same regulatory or biosynthetic pathway as the TGF-(beta) gene, is an allele of a gene member of the renin-angiotensin system (RAS).

22. The method of claim 21, wherein said allele permits angiotensin II (AII) to be produced at a level sufficient to induce the production of TGF-(beta)1 at a level and/or functional activity that correlates with the development of said condition.

23. The method of claim 21, wherein said allele is an angiotensinogen (AT) allele.

24. The method of claim 23, wherein said AT allele comprises a polymorphism within its promoter region.

25. The method of claim 23, wherein said AT allele comprises an adenine nucleotide six bases upstream from the transcription start site of AT.

26. The method of claim 23, wherein said AT allele comprises the sequence set forth in SEQ ID NO: 3.

27. The method of claim 16, wherein the fibrotic condition is selected from the group consisting of cardiac fibrosis, kidney fibrosis and hepatic fibrosis.

28. The method of claim 16, wherein the fibrotic condition is a progressive fibrosis

29. The method of claim 16, wherein the fibrotic condition is progressive hepatic fibrosis.

30. The method of claim 16, wherein the patient is infected with chronic hepatitis C virus.

31. A method for diagnosing a lower risk of developing a fibrotic condition in a patient, wherein said fibrotic condition is other than lung fibrosis, comprising detecting in a biological sample obtained from said patient a target molecule comprising an allele or an expression product thereof, wherein said allele is selected from an allele of a TGF-(beta) gene and an allele of a gene belonging to the same regulatory or biosynthetic pathway as a TGF-(beta) gene, wherein said allele correlates with said lower risk.

32. The method of claim 31, wherein said allele is a TGF-(beta)1 allele.

33. The method of claim 32, wherein said TGF-(beta)1 allele comprises a polymorphism within a signal sequence-encoding portion of the allele.

34. The method of claim 32, wherein said TGF-(beta)1 allele encodes a proline residue at codon 25 relative to the full-length open reading frame of TGF-(beta)1.

35. The method of claim 32, wherein said TGF-(beta)1 comprises the sequence set forth in SEQ ID NO: 5.

36. The method of claim 31, wherein said allele of said gene, which belongs to the same regulatory or biosynthetic pathway as the TGF-(beta) gene, is an allele of a gene member of the renin-angiotensin system (RAS).

37. The method of claim 36, wherein said allele permits angiotensin II (AII) to be produced at a level sufficient to induce the production of TGF-(beta)1 at a level and/or functional activity that correlates with the absence of said condition.

38. The method of claim 36, wherein said allele is an angiotensinogen (AT) allele.

39. The method of claim 39, wherein said AT allele comprises a polymorphism within its promoter region.

40. The method of claim 39, wherein said AT allele comprises an guanine nucleotide six bases upstream from the transcription start site of AT.

41. The method of claim 39, wherein said AT allele comprises the sequence set forth in SEQ ID NO: 6.

42. The method of claim 31, wherein the fibrotic condition is selected from the group consisting of cardiac fibrosis, kidney fibrosis and hepatic fibrosis.

43. The method of claim 31, wherein the fibrotic condition is a progressive fibrosis

44. The method of claim 31, wherein the fibrotic condition is progressive hepatic fibrosis.

45. The method of claim 31, wherein the patient is infected with chronic hepatitis C virus.

46. A method for diagnosing a higher risk of developing a fibrotic condition in a patient, wherein said fibrotic condition is other that lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein each of said different alleles correlates with said higher risk.

47. The method of claim 46, wherein said alleles are selected from a TGF-(beta)1 allele and an AT allele.

48. The method of claim 47, wherein said TGF-(beta)1 allele encodes an arginine residue at codon 25 relative to the full-length open reading frame of TGF-(beta)1.

49. The method of claim 47, wherein said AT allele comprises an adenine nucleotide six bases upstream from the transcription start site of the AT allele.

50. The method of claim 47, wherein said alleles are present in a homozygous state.

51. The method of claim 46, wherein the fibrotic condition is selected from the group consisting of cardiac fibrosis, kidney fibrosis and hepatic fibrosis.

52. The method of claim 46, wherein the fibrotic condition is a progressive fibrosis

53. The method of claim 46, wherein the fibrotic condition is progressive hepatic fibrosis.

54. The method of claim 46, wherein the patient is infected with chronic hepatitis C virus.

55. A method for diagnosing a lower risk of developing a fibrotic condition in a patient, wherein said fibrotic condition is other that lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein each of said different alleles correlates with said lower risk.

56. The method of claim 55, wherein said alleles are selected from a TGF-(beta)1 allele and an AT allele.

57. The method of claim 56, wherein said TGF-(beta)1 allele encodes a proline residue at codon 25 relative to the full-length open reading frame of TGF-(beta)1.

58. The method of claim 56, wherein said AT allele comprises a guanine nucleotide six bases upstream from the transcription start site of the AT allele.

59. The method of claim 56, wherein said alleles are present in a homozygous state.

60. The method of claim 55, wherein the fibrotic condition is selected from the group consisting of cardiac fibrosis, kidney fibrosis and hepatic fibrosis.

61. The method of claim 55, wherein the fibrotic condition is a progressive fibrosis

62. The method of claim 55, wherein the fibrotic condition is progressive hepatic fibrosis.

63. The method of claim 55, wherein the patient is infected with chronic hepatitis C virus.

64. A method for diagnosing an intermediate risk of developing a fibrotic condition in a patient, wherein said fibrotic condition is other than lung fibrosis, comprising detecting in a biological sample obtained from said patient at least two target molecules selected from different alleles of a TGF-(beta) gene or expression products thereof, and different alleles of a gene belonging to the same regulatory or biosynthetic pathway as a TGF-(beta) gene or expression products thereof, wherein at least one of said different alleles correlates with a higher risk of developing said condition and wherein at least one other of said different alleles correlates with a lower risk of developing said condition.

65. The method of claim 64, wherein said alleles that correlate with a higher risk of developing said condition are selected from a TGF-(beta)1 allele that encodes an arginine residue at codon 25 relative to the full-length open reading frame of TGF-(beta)1, and an AT allele that comprises an adenine nucleotide six bases upstream from the transcription start site of the AT allele.

66. The method of claim 64, wherein said alleles that correlate with a lower risk of developing said condition are selected from a TGF-(beta)1 allele that encodes a proline residue at codon 25 relative to the full-length open reading frame of TGF-(beta)1, and an AT allele that comprises a guanine nucleotide six bases upstream from the transcription start site of the AT allele.

67. The method of claim 64, wherein the fibrotic condition is selected from the group consisting of cardiac fibrosis, kidney fibrosis and hepatic fibrosis.

68. The method of claim 64, wherein the fibrotic condition is a progressive fibrosis

69. The method of claim 64, wherein the fibrotic condition is progressive hepatic fibrosis.

70. The method of claim 64, wherein the patient is infected with chronic hepatitis C virus.

71. A method for treating or preventing a fibrotic condition, comprising administering to a patient in need of such treatment an effective amount of an agent, which modulates the level and/or functional activity of an expression product of an allele selected from an allele of a TGF-(beta) gene and an allele of a gene belonging to the same regulatory or biosynthetic pathway as a TGF-(beta) gene, wherein said agent has been identified by a screening process comprising:
contacting a preparation comprising said expression product or a fragment of said expression product or a genetic sequence that modulates the expression of said allele with a test agent; and
detecting a change in the level and/or functional activity of said expression product or said fragment, which is indicative of an agent that is capable of effecting said modulation.

72. The method of claim 71, wherein said allele is a TGF-(beta)1 allele.

73. The method of claim 72, wherein said TGF-(beta)1 allele comprises a polymorphism within a signal sequence-encoding portion of the allele.

74. The method of claim 72, wherein said TGF-(beta)1 allele encodes an arginine residue at codon 25 relative to the full-length open reading frame of TGF-(beta)1.

75. The method of claim 72, wherein the expression product of said TGF-(beta)1 allele is a polypeptide comprising the sequence set forth in SEQ ID NO: 2.

76. The method of claim 71, wherein said allele of said gene, which belongs to the same regulatory or biosynthetic pathway as the TGF-(beta) gene, is an allele of a gene member of the renin-angiotensin system (RAS).

77. The method of claim 76, wherein said allele permits angiotensin II (AII) to be produced at a level sufficient to induce the production of TGF-(beta)1 at a level and/or functional activity that correlates with the development of said condition.

78. The method of claim 76, wherein said allele is an angiotensinogen (AT) allele.

79. The method of claim 78, wherein said AT allele comprises a polymorphism within its promoter region.

80. The method of claim 78, wherein said AT allele comprises an adenine nucleotide six bases upstream from the transcription start site of AT.

81. The method of claim 78, wherein said AT allele comprises the sequence set forth in SEQ ID NO: 3.

82. The method of claim 71, wherein the fibrotic condition is selected from the group consisting of cardiac fibrosis, kidney fibrosis and hepatic fibrosis.

83. The method of claim 71, wherein the fibrotic condition is a progressive fibrosis

84. The method of claim 71, wherein the fibrotic condition is progressive hepatic fibrosis.

85. The method of claim 71, wherein the patient is infected with chronic hepatitis C virus.


SEQUENCE LISTING (6 sequences)

SEQ ID NO: 1 - DNA, 1821 bp, Human
  5′UTR (1)..(511)
ggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgcccccaagcccacctggggccccattaaagatggagagagg

SEQ ID NO: 2 - protein, 390 aa, Human
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 3 - DNA, 1278 bp, Human
  TATA_signal (1192)..(1197)
ccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataacgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcctcgtgacccggccaggggaagaagctgccgttgttctgggtactacagcagaaggtaagccgggggccccctca

SEQ ID NO: 4 - DNA, 1821 bp, Human
  5′UTR (1)..(511)
ggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgcccccaagcccacctggggccccattaaagatggagagagg

SEQ ID NO: 5 - protein, 390 aa, Human [sequence omitted]

SEQ ID NO: 6 - DNA, 1278 bp, Human
  TATA_signal (1192)..(1197)
ccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataacgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcctcgtgacccggccgggggaagaagctgccgttgttctgggtactacagcagaaggtaagccgggggccccctca